(19) United States
(12) Patent Application Publication
(10) Pub. No.: US 2021/0253499 A1
(43) Pub. Date: Aug. 19, 2021

(54) PROCESS AND SYSTEM TO PURIFY GAS

(71) Applicant: SHELL OIL COMPANY, HOUSTON, TX (US)

(72) Inventors: Raymond Petrus Henricus Maria BERGMANN, Amsterdam (NL); John Henry SPECHT, Amsterdam (NL); Carolus Antonius Cornelis VAN DE LISDONK, Rijswijk (NL)

(21) Appl. No.: 17/261,087

(22) PCT Filed: Jul. 16, 2019
(86) PCT No.: PCT/EP2019/069125

§ 371 Date: Jan. 18, 2021

(30) Foreign Application Priority Data
Jul. 18, 2018 (EP) 18184106.5

(51) Int. Cl. C07C 7/11; B01D 53/14

(52) U.S. Cl. CPC C07C 7/11; B01D 53/1493; B01D 53/1412; B01D 53/1475; B01D 2252/504; B01D 2252/20426; B01D 2252/20431; B01D 2252/2056; B01D 2252/103

(57) ABSTRACT

A process for producing a purified gas stream from a feed gas stream comprising methane, carbon dioxide, and aromatic compounds selected from the group of benzene, toluene, ethylbenzene, o-xylene, m-xylene and p-xylene involves measuring respective amounts of carbon dioxide and aromatic compounds in the feed gas and providing the measured amounts to a controller. The feed gas stream and a stream of absorbing liquid comprising at least sulfolane, water and a secondary or tertiary amine is provided to an acid gas removal unit (AGRU) The controller adjusts one or more of composition, temperature, and flow rate of the stream of absorbing liquid to the AGRU to prevent amounts of aromatic compounds in an AGRU outlet stream from exceeding a predetermined maximum threshold.

### FIELD OF THE INVENTION

The present invention is directed to a process and a system to purify gas. The gas can be natural gas directly from a well, or pipeline gas which has typically been pretreated. The purified gas may be suitable to be liquefied.

### BACKGROUND TO THE INVENTION

Gas streams from natural gas wells typically comprise contaminants such as carbon dioxide, hydrogen sulphide, and aromatic hydrocarbons such as benzene, toluene, ethylbenzene, and xylene that need to be removed before the gas streams can be further used.

A recent industry development is the use of pipeline gas, rather than natural gas, as the feed source for liquefied natural gas (LNG) projects. Despite the limited amount of heavier hydrocarbon components in this feed gas, pipeline-gas projects have continued to require a natural gas liquids (NGL) extraction unit in the line-up as it performs another critical function: deep benzene, toluene, ethylbenzene and xylene (BTEX) removal.

Processes for removing hydrogen sulfide, carbon dioxide and aromatic hydrocarbons from a gas stream typically comprise an absorption step for removing hydrogen sulfide, carbon dioxide and aromatic hydrocarbons from the gaseous feed stream by contacting such gaseous feed stream with a solvent, for example an amine solvent, in an absorption column. Thus a purified gaseous stream is obtained and a solvent loaded with contaminants. The loaded solvent is typically regenerated in a stripper to obtain a gas stream comprising contaminants and a lean solvent that is recycled to the absorption column.

BTX is often used as acronym for benzene, toluene, and xylenes (e.g. o-xylene, m-xylene and/or p-xylene). BTEX is often used as acronym for benzene, toluene, ethylbenzene, and xylenes (e.g. o-xylene, m-xylene and/or p-xylene).

When producing LNG (liquefied natural gas), BTEX is removed prior to liquefaction to avoid freezing. When the level of BTEX in the gas is too high, tubes may become plugged during liquefaction. Generally it is preferred to have a BTEX concentration in the gas of at most 3 ppmv (parts per million by volume) before liquefaction.

Like an increasing number of recent other LNG opportunities, especially in areas that have abundant quantities of domestic gas such as North America, Canada, Russia and North Africa, pipeline gas has been used for LNG production. Pipeline gas hastypically been treated to gas grid specifications. The composition of the gas will therefore differ from natural gas direct from the producing wells.

Pipeline gas typically comprises, in addition to methane, low levels of carbon dioxide (CO2) (in the range of about 0.5 to 2.0 mol %) and small amounts of other hydrocarbons, most notably BTEX (in the range of for instance 25 to 250 ppmv). Pipeline gas has been hydrocarbon dewpointed for transport in the pipeline, and the quantity of heavier hydrocarbons does not warrant the investment in process facilities and infrastructure to recover these heavier hydrocarbons as liquids.

In an LNG plant, it is imperative that the feed to the LNG cryogenic block meets stringent BTEX specifications. These components would freeze when the methane is liquefied, leading to plugging of equipment and, in turn, a plant shutdown and lost production. It is therefore important for the project engineers to have confidence that their configuration will be able to remove these components from the gas to the desired specification.

WO2007003618 describes a process in which benzene, toluene, o-xylene, m-xylene and p-xylene (BTX) are removed by means of an absorbing liquid comprising a physical solvent. In this step, hydrogen sulfide and carbon dioxide are also removed to a large extent. A mixture of sulfolane, a secondary or tertiary amine, and water can be used as absorbing liquid. After benzene, toluene, and xylenes have been removed by means of an absorbing liquid, the concentration is reduced further. This is typically performed by means of a scrubber column, an adsorber, an extraction unit, or another type of BTEX or BTX removal unit. In such a second step it is sometimes also possible to remove hydrocarbons with more than 5 carbon atoms (C5+) from the gas.

WO2016150827 discloses a process improving the efficiency of the removal of benzene, toluene, and xylenes by contacting the gas with a specific absorption liquid and reducing the temperature in the absorption column. The process comprises the steps of (a) contacting the feed gas stream with absorbing liquid comprising sulfolane and a secondary or tertiary amine in an absorption column, and reducing the temperature of the absorbing liquid at an intermediate section of the absorption column, to obtain loaded absorbing liquid comprising carbon dioxide, hydrogen sulphide, and aromatic compounds selected from the group of benzene, toluene, o-xylene, m-xylene and p-xylene, and a gas stream depleted of these compounds; and (b) cooling and de-pressurizing at least a part of the gas stream obtained in step (a) to obtain a liquid comprising aromatic compounds selected from the group of benzene, toluene, ethylbenzene, o-xylene, m-xylene and p-xylene, and flash gas depleted of these compounds.

According to WO2016150827, step (a) of the disclosed process is able to reduce the mol % of BTX in a feed gas up to 40%. However, for instance to be able to handle the wide range of impurities in pipeline gas, additional equipment and process steps is required to meet the impurities specification for subsequent liquefaction. For instance, the process of WO2016150827 requires a separate flash unit to remove BTX to below a set threshold, thus increasing equipment costs. Herein, capital expenditure typically is key to the economic viability of a project for processing gas.

WO-2017/137309 provides a method for separating C5-C8 hydrocarbons and acid gases from a fluid stream. The method of WO-2017/137309 cannot predict or guarantee outlet concentration for aromatic or soluble components. WO-2017/137309 is directed to using a heated flash to dispose of aromatic and hydrocarbon components.

WO2007/003618 provides a process for producing a gas stream depleted of RSH from a feed gas comprising natural gas, RSH and aromatic compounds selected from the group of benzene, toluene, o-xylene, m-xylene and p-xylene. The concentration of BTX compounds in the gas stream obtained in a first step of the process (step (a)) depends on the concentration of these compounds in the feed gas stream.

### SUMMARY OF THE INVENTION

It is an aim to provide a more robust process to purify a gas. More robust herein may include the ability to handle a wider range of impurities in the feed stream and/or to do so at lower cost.

In one aspect, the present invention is directed to a process for producing a purified gas stream from a feed gas stream comprising methane, carbon dioxide, and aromatic compounds selected from the group of benzene, toluene, ethylbenzene, o-xylene, m-xylene and p-xylene (BTEX), the process comprising the steps of:
measuring respective amounts of carbon dioxide and aromatic compounds in the feed gas;
providing the measured amounts of carbon dioxide and aromatic compounds in the feed gas to a controller;
providing the feed gas stream to an acid gas removal unit (AGRU);
providing a stream of absorbing liquid comprising at least sulfolane, water and a secondary or tertiary amine to the AGRU for contacting the feed gas stream with the absorbing liquid in the AGRU;
providing an AGRU waste stream comprising absorbing liquid loaded with carbon dioxide and aromatic compounds;
providing an AGRU outlet stream wherein carbon dioxide and aromatic compounds have been at least partially removed; and
the controller adjusting one or more of composition, temperature, and flow rate of the stream of absorbing liquid to the AGRU to prevent amounts of aromatic compounds in the AGRU outlet stream from exceeding a predetermined maximum threshold.

The process of the disclosure is relatively robust. The process enables an increased window of operation with respect to impurities in the feed gas stream for given predetermined design specifics of the equipment.

In an embodiment, the method comprises the steps of:
measuring respective amounts of carbon dioxide (CO2) and aromatic compounds in the AGRU outlet stream;
providing the measured amounts of carbon dioxide and aromatic compounds in the AGRU outlet stream to the controller; and
the controller adjusting one or more of composition, temperature, and flow rate of the stream of absorbing liquid to the AGRU to prevent said measured amounts of aromatic compounds in the AGRU outlet stream from exceeding the predetermined maximum threshold.

In another embodiment, the method comprises the step of guaranteeing removal of aromatic compounds down to said predetermined maximum threshold in the AGRU outlet stream.

In yet another embodiment, the method comprises the step of guaranteeing an AGRU output stream comprising a total amount of BTEX of 3 or 4 ppmv or less.

Said maximum threshold for aromatic compounds may be 3 ppmv benzene and less than 3 ppmv toluene, ethylbenzene and xylene.

In an embodiment, the process includes creating a model of the AGRU to predict removal of the aromatic compounds from the AGRU outlet stream based on the solubility of the aromatic compounds in the absorbing liquid.

In an embodiment, the model includes dependency of the BTEX removal on one of more of absorbing liquid flow rate, absorbing liquid temperature, CO2 content of the feed gas stream, BTEX content in the feed gas, feed gas flow rate, feed gas temperature, AGRU absorber size, and absorbing liquid composition.

In an embodiment, the model includes the steps of:
performing thermodynamic measurements of hydrocarbon solubility in the absorbing liquid to provide experimental data;
modelling hydrocarbon solubility in the absorbing liquid based on the experimental data to provide a hydrocarbon solubility model;
including the hydrocarbon solubility model in an overarching rate-based mass transfer model;
obtaining measurements of AGRU operation at one or more operational sites top provide operational measurement data;
validating the mass transfer model against the operational measurement data;
deriving a model describing the impact of process parameters on the removal of soluble components in the AGRU;
applying a design and control philosophy to the AGRU;
correcting the design and control philosophy using operational data; and
implementing the design and control philosophy in the AGRU.

In another embodiment, the method comprises the step of providing the AGRU outlet stream to a molsieve.

In an embodiment, the process includes the steps of:
providing a molsieve outlet stream from the molsieve to a cooler for providing a cooled molsieve outlet stream; and
providing the cooled molsieve outlet stream to a flash unit.

In yet another embodiment, the process comprises the step of depressurizing the cooled molsieve outlet stream to provide a depressurized molsieve outlet stream to the flash unit.

In an embodiment, the process comprises the step of contacting the cooled molsieve outlet stream or the depressurized molsieve outlet stream with a wash liquid in the flash unit to provide a flash gas stream.

In an embodiment, the process comprises the step of compressing the flash gas stream to provide a compressed flash gas stream.

In an embodiment, the process comprises the steps of:
measuring respective amounts of carbon dioxide (CO2) and aromatic compounds in the AGRU outlet stream;
providing the measured amounts of carbon dioxide and aromatic compounds in the AGRU outlet stream to the controller; and
the controller adjusting one or more of composition, temperature, and flow rate of the stream of absorbing liquid to the AGRU to prevent said measured amounts of aromatic compounds in the AGRU outlet stream from exceeding the predetermined maximum threshold.

According to another aspect, the disclosure provides a system for producing a purified gas stream from a feed gas stream comprising methane, carbon dioxide, hydrogen sulfide, and aromatic compounds selected from the group of benzene, toluene, ethylbenzene, o-xylene, m-xylene and p-xylene (BTEX), the system comprising:
a first measurement device for measuring respective amounts of carbon dioxide and aromatic compounds in the feed gas stream;
a controller coupled to the measurement device and arranged to receive the measured amounts of carbon dioxide and aromatic compounds in the feed gas;
an acid gas removal unit (AGRU) for receiving the feed gas stream and adapted for contacting the feed gas stream with a stream of absorbing liquid comprising at least sulfolane, water and a secondary or tertiary amine, the AGRU being adapted to provide an AGRU waste stream comprising absorbing liquid loaded with carbon dioxide and aromatic compounds, and an AGRU outlet stream wherein carbon dioxide and aromatic compounds have been at least partially been removed;
the controller being adapted to adjust one or more of composition temperature, and flow rate of the stream of absorbing liquid to the AGRU to prevent amounts of aromatic compounds in the AGRU outlet stream from exceeding a predetermined maximum threshold.

The system is relatively robust. The system enables an increased window of operation with respect to impurities in the feed gas stream for given predetermined design specifics and characteristics of the equipment comprised in the system.

In an embodiment, the system comprises:
a second measurement device measuring respective amounts of carbon dioxide and aromatic compounds in the AGRU outlet stream, adapted for providing the measured amounts of carbon dioxide and aromatic compounds in the AGRU outlet stream to the controller.

### BRIEF DESCRIPTION OF THE DRAWINGS

The drawing figures depict one or more implementations in accord with the present teachings, by way of example only, not by way of limitation. In the figures, like reference numerals refer to the same or similar elements. Herein:

FIG. 1 shows an exemplary diagram of an embodiment of a system according to the present disclosure;

FIG. 2 shows a cross sectional diagram of an embodiment of an absorption column according to the present disclosure;

FIG. 3 shows a cross sectional diagram of another embodiment of an absorption column according to the present disclosure;

FIG. 4 shows a diagram of an embodiment of a system according to the present disclosure;

FIG. 5 shows a diagram of another embodiment of a system according to the present disclosure; and

FIG. 6 shows a diagram of a conventional lineup;

FIG. 7 shows a diagram of an embodiment of a system according to the present disclosure;

FIG. 8 shows a diagram of another embodiment of a system according to the present disclosure;

FIGS. 9-11 show exemplary diagrams indicating control of an operating window according to a process of the present disclosure; and

FIG. 12 shows a diagram of an exemplary model included in an embodiment of the method or system of the present disclosure.

### DETAILED DESCRIPTION OF THE INVENTION

Certain terms used herein are defined as follows:

(Feed) gas stream may encompass any stream of (feed) gas, including but not limited to pipeline gas and natural gas.

The gas stream may comprise methane. In addition, the gas stream may comprise carbon dioxide, hydrogen sulphide, and/or aromatic compounds selected from the group of benzene, toluene, ethylbenzene, o-xylene, m-xylene and p-xylene. In addition, the gas stream may comprise hydrocarbons with more than 5 carbon atoms (C5+).

Natural gas is a general term that may refer to mixtures of light hydrocarbons and optionally other gases (nitrogen, carbon dioxide, helium) derived from natural gas wells. The main component of natural gas is methane. In addition to methane, natural gas may comprise higher hydrocarbons, such as ethane, propane and butane. In some cases (small) amounts of heavier hydrocarbons may be comprised in the natural gas, often indicated as natural gas liquids or condensates. When produced together with oil, the natural gas may be referred to as associated gas. Other compounds that may be present as contaminants in natural gas in varying amounts include carbon dioxide, hydrogen sulphide, and aromatic compounds.

The feed gas stream may comprise H2S, for example in the range between 0 to about 10 vol % or more, based on the total feed gas stream. The feed gas stream may also comprise carbon dioxide, for example in the range from 0 to about 40 vol %, based on the total feed gas stream.

A line-up for liquefying a feed gas 10 may comprise a separator 14 and a pre-heater or cooler 16. The lineup features an acid gas removal unit (AGRU) 17, for removing CO2 and/or hydrogen sulphide (H2S). The AGRU 17 may comprise an absorber 18. The absorber 18 may be coupled to a regenerator 22 (which is also part of AGRU 17) for producing AGRU waste stream 24.

An AGRU output stream 26 may be forwarded to a molecular sieve (molsieve) 20 for dehydration of the AGRU output stream 26. A molsieve waste stream 21 may be provided to a regenerator 32, for providing a regenerator output stream 33 to a two-phase separator 34. The separator 34 outputs a vapor stream 36 and a liquid stream 38. Molsieve output stream 29 may be provided to the pre-cooler 30.

Following the pre-cooler 30, a conventional lineup may typically comprise an NGL extraction and fractionation unit, which removes BTEX and C5+ in an NGL waste stream. A second pre-cooler 44 may be connected to an outlet of the NGL extraction unit. This allows the conventional system to provide pretreated feed gas 46 to the main cryogenic heat exchanger (MCHE).

However, pipeline gas has already been dewpointed so the gas has relatively low levels of hydrocarbons beyond methane (C2+) and there is little economic driver to install the NGL extraction kit (pre-liquefaction plus fractionation) necessary to recover liquids. The NGL extraction kit is relatively capital intensive as it requires multiple columns and auxiliary equipment. Obviating the NGL extraction unit therefore can have a major positive impact on the economics of a project.

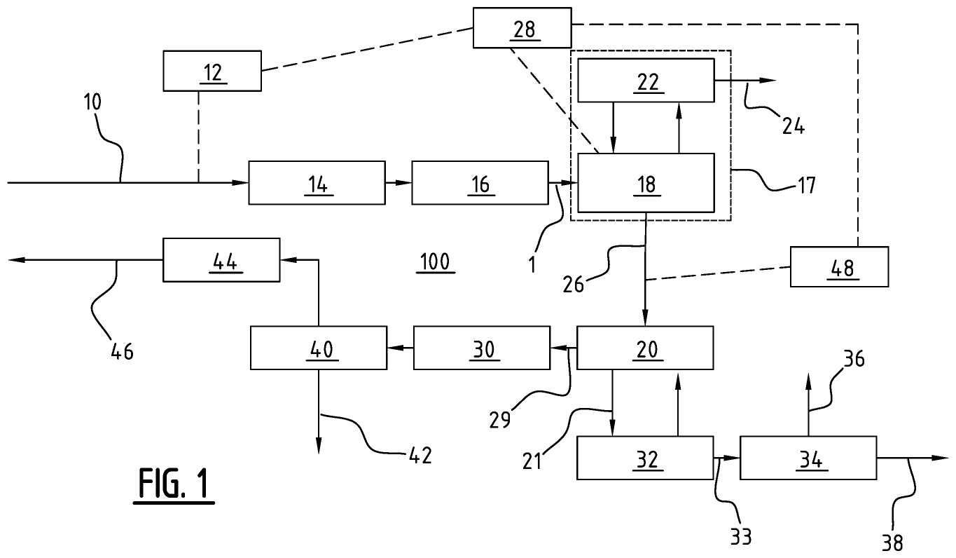

FIG. 1 shows an embodiment of a system 100 proposed by applicants. The system 100 features two main differences with respect to a conventional lineup. First, an AGRU solvent, typically ADIP-X, is replaced by Sulfinol. Sulfinol is a hybrid solvent, consisting of an aqueous amine and an additional component, sulfolane, which provides enhanced physical solubility of certain components. Unlike ADIP-X, it can also remove BTEX. The system and process of the present disclosure allow optimization, such that the AGRU 17 already removes BT(E)X to the required specifications. In other words, the process or system of the present disclosure enable the AGRU 17 to provide an AGRU output stream 26 which is already within predetermined BTEX specifications suitable for liquefying gas. In other words, the method and system of the disclosure allow the AGRU outlet stream 26 to already meet the BTEX specifications for the downstream liquefying process. In the conventional system, these specifications were not met until the flash outlet stream 46.

BTEX components can be removed from the feed gas by the AGRU, and can be comprised in AGRU waste stream 24. The specifications for this removal are, for instance, a maximum threshold for BTEX in the AGRU outlet stream 26. Said maximum threshold may be, for instance, about 10 ppmv BTEX or less. More preferably, the system and method can guarantee an AGRU output stream 26 comprising about 3 or 4 ppmv total amount of BTEX or less. In a practical embodiment, the system and method of the disclosure can guarantee removal of contaminants in the AGRU outlet stream to below 1 ppmv of total amount of BTEX or less (down to traces of BTEX left). The required BTEX specification will be set by the downstream equipment.

The system may comprise a first measurement device 12 to measure or sample the composition of the feed gas stream 10. The first measurement device may be a sensor to measure online. In a practical embodiment, the first measurement device 12 may be a sampler for taking a sample from the feed gas 10, for instance at predetermined intervals. The composition of said samples may be examined offline in a lab. The samples may be taken periodically or periodically at preset time intervals. A suitable time interval may be in the order of a month or a year.

The system may comprise an optional second measurement device 48 to measure or sample the composition of the AGRU outlet stream 26. The second measurement device may be a sensor to measure online. In a practical embodiment, the second measurement device 48 may be a sampler for taking a sample from the AGRU outlet stream 26, for instance at predetermined intervals. The composition of said samples may be examined offline in a lab. The samples may be taken periodically or periodically at preset time intervals. A suitable time interval may be in the order of a month or a year.

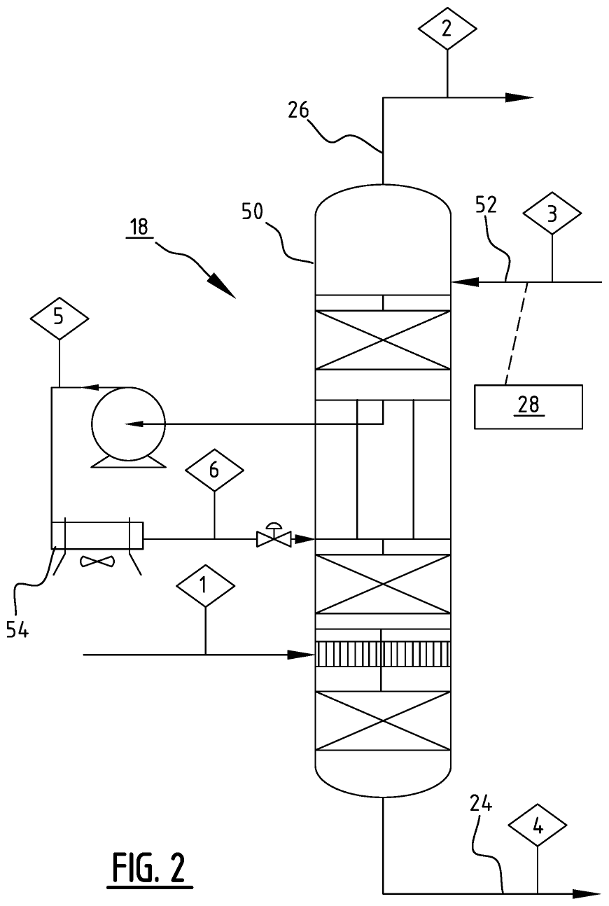

The system may comprise a controller 28 for controlling the AGRU 17 in response to the measured feed gas composition and/or the measured AGRU outlet stream composition. The controller 28 may for instance adjust the composition, temperature and/or the flow rate of the solvent entering the absorber 18 of the AGRU (see FIG. 2). The solvent may comprise Sulfinol. Thus, the controller 28 can optimize the gas treating requirements within the required capital costs and operating expenses of the AGRU 17.

Sulfolane (also tetramethylene sulfone, systematic name: 2,3,4,5-tetrahydrothiophene-1,1-dioxide) is an organosulfur compound, formally a cyclic sulfone, with the formula (CH2)4SO2. It is a colorless liquid commonly used in the chemical industry as a solvent for extractive distillation and chemical reactions. Sulfolane was originally developed by the Shell Oil Company in the 1960s as a solvent to purify butadiene. Sulfolane is a polar aprotic solvent, and it is readily soluble in water. Sulfolane, a component of Sulfinol, can remove the aromatic compounds and remnants of mercaptans and other organic sulphur compounds which may remain in the pipeline gas.

Despite its enhanced capability, the AGRU is not significantly more expensive in this line-up, it is just loaded with a different solvent. The high-pressure equipment dimensions are primarily set by the gas flow rate of the feed gas 10.

A second change is replacing the NGL extraction unit with a considerably less-expensive cold flash 40. In the process of the present disclosure, the BTEX specification can be met in the AGRU 17. The optional flash unit 40 can remove other heavy hydrocarbons. Thus, the flash unit 40 can ensure that the pretreated feed gas 46 meets a predetermined C5+ specification (for instance up to a maximum of 0.1 mol %). The flash unit 40 is optional, and can be omitted on projects that have a less stringent C5+ specification to meet, or a leaner feed gas composition. In the system and method of the disclosure, the AGRU 17 can already meet BTEX specifications. Removing the flash unit 40 further enhances the capital efficiency of this line-up. The pretreated gas 46 is suitable for, for instance, subsequent liquefying in a (main) cryogenic heat exchanger MCHE (not shown). On the other hand, the optional flash unit 40 can remove additional BTEX from the gas stream. The latter allows the specification at the AGRU outlet to be relaxed.

FIG. 1 shows an exemplary line-up including the AGRU 17, comprising absorber unit 18 and recovery unit 22. The Sulfinol solvent in the AGRU removes the BTEX in addition to CO2 and H2S. The outlet stream 26 of the AGRU 17 can have BT(E)X levels removed to at or below a predetermined threshold specification. Remaining heavier hydrocarbons—typically at least C₅₊— can be removed by an optional cold flash 40, which replaces the capital-intensive NGL extraction unit.

Project-specific calculations show that, the line-up shown in FIG. 1 could reduce the installed capital cost (upfront equipment costs) by more than $100 million. Further, the equipment count for the system shown in FIG. 1 would also be reduced, for instance by as much as 50%.

In addition to the capital cost savings, the proposed line-up would also provide operational cost savings. For example, the compression power required to run the NGL extraction unit would be avoided.

While the capital and operational cost savings are attractive, a typical LNG facility requires certainty that the line-up can achieve the required maximum BTEX level before entering the MCHE. The lineup 100 of the present disclosure can be able to guarantee a maximum level of BTEX in the AGRU outlet stream 26. Said maximum level may be, for instance, about 3 ppm down to 1 ppm post AGRU (i.e. in the AGRU outlet stream 26). Alternatively, the system 100 can guarantee a maximum level of BTEX in the pretreated gas stream 46 provided to the MCHE. Further, the system 100 is preferably sufficiently robust to be able to handle a relatively wide range of impurities, due to changes in feed gas composition and operational uncertainties and/or variations.

Guaranteeing the level of BTEX in the AGRU outlet stream 26 can be controlled by controlling the solvent flow in the AGRU. In effect, this indicates that the feed flow rate, i.e. the flow rate of the feed gas 10, can be maintained at the design capacity and reduction of the feed flow rate can be obviated even in case the feed gas comprises more BTEX than anticipated in the design phase. Thus, the system and method of the disclosure allow to guarantee BTEX specifications either in the AGRU outlet stream or, at least, in the flash unit outlet stream 46, while maintaining the feed gas flow rate at or above a predetermined design flow rate. The latter enables, for instance, one or more of optimizing rate of production, ensuring to meet obligations for delivery, etc.

To estimate BTEX removal, Applicant's gas processing group has embarked on a significant R&D program to improve its understanding of BTEX solubility in solvents comprising sulfolane. This involved comprehensive dedicated thermodynamic experiments that explored the solubility of BTEX components in the solvent in the presence of CO2. CO2 will typically also be comprised to some extent in the feed gas 10. Tests and calculations were done at various temperatures of up to 90° C. These experiments were then used to derive a physical model for the removal of benzene and other BTEX components in the AGRU. This model was then validated against field data from Applicant's operations. The model provided a highly accurate portrayal of the behavior of the BTEX within the AGRU. This model was combined with a robust design methodology which now enables Applicant to design the AGRU unit absorber 18 with high confidence of the BTEX behavior in order to guarantee that it can meet the required outlet specifications. Specifications may be set at a maximum of, for instance, 3, or even down to 1, ppmv for BTEX.

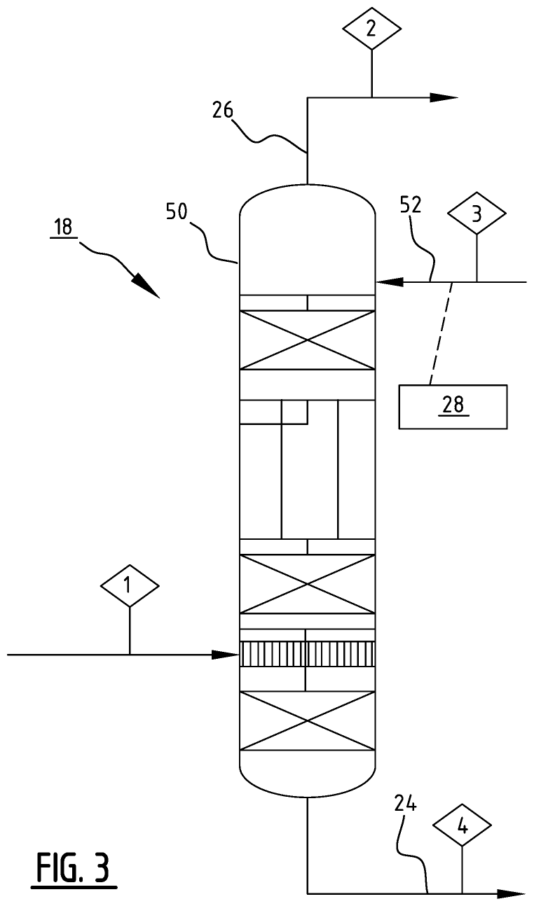

The design is also robust against fluctuations in, for example, the feed gas composition 10, the sulfolane concentration in the solvent and operating temperatures. So for example, if the BTEX concentration of the feed gas 10 to the AGRU increases, Applicant's modelling tool can advise by how much the flow rate and/or composition of solvent 3 should be adjusted (See FIGS. 2 and 3).

A comparison of the capital cost of the line-up of FIG. 1 versus a conventional line-up on an archetypal project may be in the order of 10 to 20% reduction in installed capital cost for the proposed line-up, compared with the base case. The installed capital cost of the equipment could provide a saving in the order of 50 to 100 million USD. This is based on the gas conditions of the treated feed gas 46 suitable for the MCHE. The latter has, for instance, the following treatment specifications: benzene less than 3 ppmv, toluene, ethylbenzene and xylene less than 4 ppmv; C5+ less than 500 ppmv; and CO2 less than 50 ppmv.

These insights mean that other greenfield LNG projects using pipeline gas as the feed could achieve capital cost savings of a similar order of magnitude. As a result of its efforts to improve and validate the behavior of BTEX in solvents, the system and method of the present disclosure can guarantee removal of BTEX down to 3 ppmv benzene and less than 3 ppmv toluene, ethylbenzene and xylene. Said guarantee can apply to the AGRU outlet stream 26. At least, said guarantee applies to the pretreated gas stream 46.

FIG. 2 shows an example of an absorption column 50 of an AGRU 17 suitable for the system and method of the present disclosure. The column 50 is provided with optional inter-stage cooling. A feed gas stream 1 enters the absorption column 50 via a suitable conduit. Cleaned AGRU outlet stream 26 leaves the absorption column 50 via top outlet line 2. Absorbing liquid 52 is provided to the absorption column 50 via absorbent inlet line 3.

AGRU waste stream 24 leaves the absorption column 50 via lower end outlet line 4. The AGRU waste stream 24 herein comprises solvent comprising impurities absorbed from the feed gas stream 1. Said impurities may include one or more of carbon dioxide, hydrogen sulphide, and aromatic compounds selected from the group of benzene, toluene, o-xylene, m-xylene and p-xylene.

The absorbing liquid 52 entering via line 3 does not comprise or is lean with regard to carbon dioxide, hydrogen sulphide, and aromatic compounds selected from the group of benzene, toluene, o-xylene, m-xylene and p-xylene.

The absorbing liquid 52 enters the column 50 near a top end thereof. The absorption of carbon dioxide is exothermal and the temperature of the absorbing liquid increases while flowing down within the column 50. Optionally, warm absorbing liquid 5 may be removed from the absorption column 50 and after cooling in intercooler 54, cooled absorption liquid 6 is fed back to the absorption column 50.

FIG. 3 shows an absorption column 50 without intercooler. In a system according to the present disclosure, the intercooler may be obviated, reducing equipment costs.

The absorbing liquid 52 removes contaminants by transferring contaminants included in the feed gas stream 1 to the absorbing liquid. This results in an absorbing liquid loaded with contaminants. The loaded absorbing liquid 24, comprising said contaminants, may be regenerated by contacting with a regeneration gas.

In a practical embodiment, the absorbing liquid 52 at least comprises sulfolane. In addition, the absorbing liquid may comprise a secondary or tertiary amine. Sulfolane is a physical solvent. The secondary or tertiary amine is a chemical solvent. In a practical embodiment, the absorbing liquid 52 additionally comprises another solvent, such as water.

The amount of sulfolane in the absorbing liquid 52 may vary, for instance in the range of from 10 to 60 parts by weight based on the total volume of the absorbing liquid 52. In an embodiment, the amount of sulfolane is varied between 15 to 50, more preferably from 20 to 40 parts by weight, based on the total volume of the absorbing liquid. The remainder of the absorbing liquid is secondary or tertiary amine and suitably another solvent, such as water.

Examples of suitable secondary or tertiary amines are an amine compound derived from ethanol amine, such as DIPA (di-isopropanolamine), DEA, MMEA (monomethyl-ethanolamine), MDEA, or DEMEA (diethyl-monoethanolamine), preferably DIPA or MDEA, most preferably MDEA.

The absorbing liquid may further comprise a so-called activator compound. Suitable activator compounds are piperazine, methyl-ethanolamine, or (2 aminoethyl)-ethanolamine, especially piperazine.

The absorbing liquid typically comprises water, preferably in the range of from 15 to 45 parts by weight, more preferably of from 15 to 40 parts by weight of water.

Optionally, the temperature of the absorbing liquid is reduced at an intermediate section of the absorption column 50. The temperature of the absorbing liquid may be reduced by means of removing absorbing liquid 5 from the absorption column, cooling the removed absorbing liquid using cooler 54, and feeding cooled absorbing liquid 6 back to the absorbing column. Cooled liquid 6 may be fed back to the absorbing column 50 at a level lower than at which warmed absorbing liquid 5 is removed from the absorbing column. But the cooled liquid 6 can also be fed back at the same level, or at a level higher than the level whereat the warmed absorbing liquid 5 is removed from the absorbing column 50.

The temperature of the absorbing liquid can optionally be reduced by means of inter-stage cooling. The temperature of the absorbing liquid may be reduced by means of an intercooler 54. An intercooler can be obtained, for example, from Black & Veatch. Interco® ling typically happens to a temperature 10 to 30 degrees below the temperature of the solvent into the intercooler. The temperature of the cooled solvent remains positive (above 0 degree C.), typically around 30 degree C.

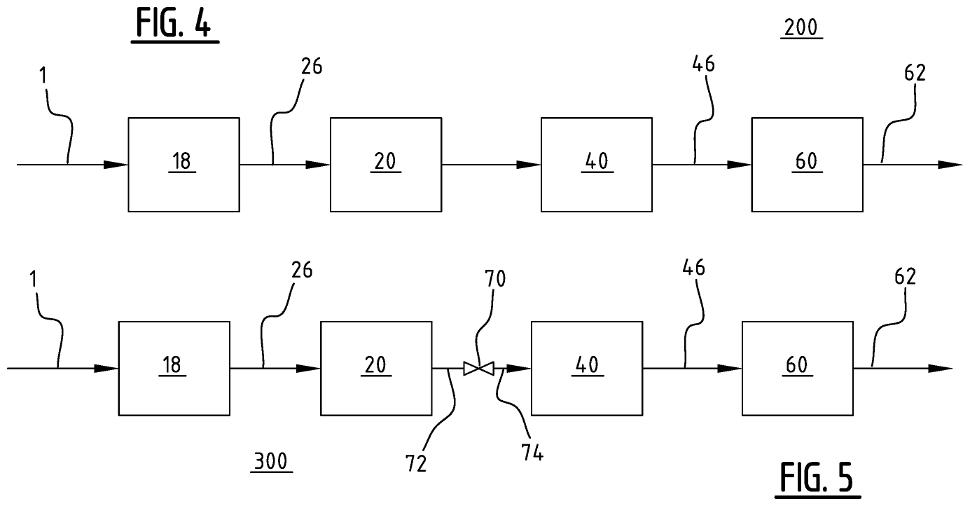

FIG. 4 shows an exemplary embodiment of system 200 according to the present disclosure. Herein, AGRU 17 lacks an intercooler. Cold flash unit 40 lacks a washing step. Optionally, the system 200 is provided with compressor 60 to compress pretreated gas 46 before sending compressed pretreated gas 62 to the MCHE. Also, the system 200 obviates a depressurization step between the molsieve 20 and the cold flash 40.

FIG. 5 shows another embodiment of system 300 according to the present disclosure. Herein, AGRU 17 may be provided with an intercooler 54 (as shown in FIG. 2). Alternatively, AGRU may lack an intercooler. Cold flash unit 40 may be provided with a washing step. Alternatively, cold flash unit 40 may lack a washing step. Optionally, the lineup 300 comprises a compressor 60 to compress the treated gas 46 before sending the treated and compressed gas 62 to the MCHE. Optionally, the system 300 comprises a depressurization valve 70 between the molsieve 20 and the cold flash unit 40. The depressurization valve 70 may be arranged between the precooler 30 and the cold flash 40 (see also FIG. 8). The depressurization valve 70 depressurizes the gas outlet stream 72 as received from the molsieve 20 and/or from the precooler 30. Depressurized gas stream (typically depressurized and cooled gas stream) 74 is provided from the valve 70 to the cold flash unit 40.

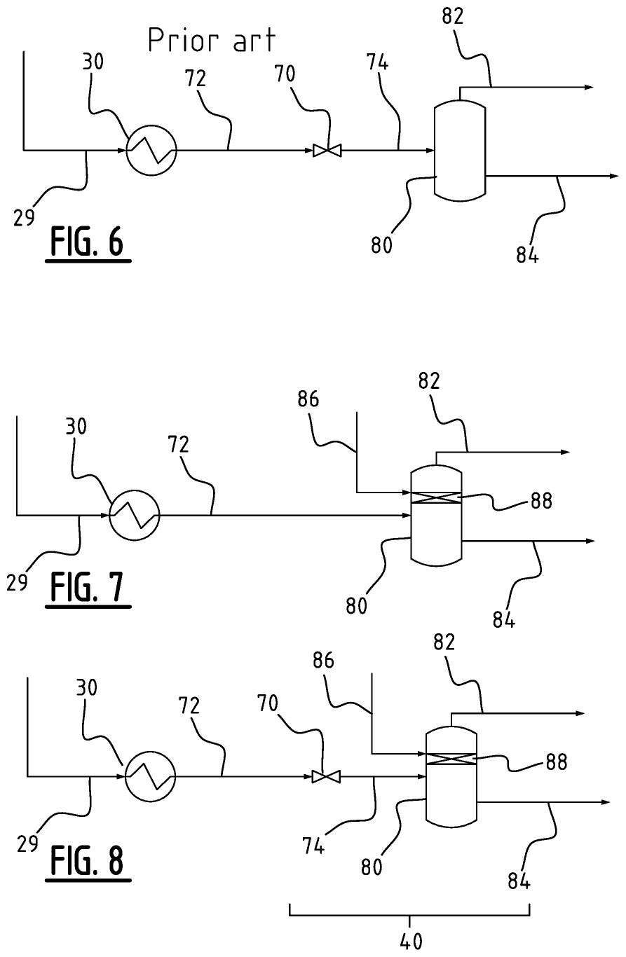

FIGS. 6 to 8 depict various lineups of the precooler unit 30 and the cold flash unit 40 respectively in more detail.

FIG. 6 shows a lineup disclosed in WO2007003618, which includes the pre-cooler 30 followed by depressurization valve 70 and a separator 80. The separator vessel 80 separates liquid and vapor components. The separator 80 is part of the cold flash unit 40 (FIG. 1). Vapor stream 82 is provided at a top end outlet of the separator vessel. Liquid stream 84 is provided at a lower end outlet of the separator 80. Herein, the cold flash is included to further remove BTEX and/or C5+ components from the process stream.

FIG. 7 shows an embodiment of a lineup including the pre-cooler 30. This lineup lacks a depressurization valve 70. Pressurized and precooled gas stream 72 is provided to the separator 80. The separator vessel 80 separates liquid and vapor components. Vapor stream 82 may be provided at a top end outlet of the separator vessel. Liquid stream 84 is provided at a lower end outlet of the separator 80. The separator is provided with a wash system. Herein, a wash stream 86 is provided to a wash section or tray section 88 included in the separator 80.

FIG. 8 shows another embodiment of a lineup including the pre-cooler 30 and a depressurization valve 70. Pressurized and precooled gas stream 72 is provided to the optional depressurization valve 70. Pre-cooled and depressurized gas stream 74 is provided to the separator 80. The separator may be provided with a wash system. Herein, a wash stream 86 is provided to a wash section or tray section 88 included in the separator 80.

The wash stream 86 may comprise liquid hydrocarbons. The wash liquid can comprise, for instance, propane, butane and/or other C3+ hydrocarbons. These C3+ hydrocarbons can originate from stabilized condensate or from a fractionation unit.

FIG. 7 and FIG. 8 show a cold flash section 40 including a wash section 88. The wash section 88 can remove BTEX even further. The wash section can also be designed to remove C5+ components from the process stream.

FIG. 8 optionally includes a pressure let down (depressurization valve 70) which increases the ability for knocking out C5+ and BTEX in the separator 80.

FIG. 7 shows a preferred embodiment, which will allow to design for sufficient removal of BTEX in the AGRU 17 combined with removal of C5+ in the flash unit 40. Herein, the cooling by precooler 30 can be designed to remove C5+ to below a predetermined threshold (the threshold being for instance about 1000 ppm or below in the outlet stream 46).

A method of the present disclosure may include the step of modelling the AGRU unit 17 to provide a measure for the removal of impurities from the gas stream 10. The model may be based on measured data. Said measured data may comprise, at least, one or more of: Temperature data at various locations in the AGRU and/or at the inlet and outlet of the AGRU; Compositional data of the gas at the inlet of the AGRU; and measured data on the solubility of impurities in the solvent for varying solvent composition, solvent temperature, feed gas composition, and/or feed gas flow rate; solubility of impurities in the presence of predetermined additional components, such as CO2.

The model of the method may be based on new temperature data, and updated solubility correlations for the solvent. Using the model allows the method and system of the present disclosure to guarantee BTEX removal. The method allows to correct for feed gas variations, for instance by controlling the feed gas flow rate and temperature, the solvent flow rate, the solvent temperature and/or the solvent composition. The guarantee works down to 1 ppmv BTEX in the AGRU outlet stream 26, irrespective of inlet conditions up to a maximum BTEX threshold. Said maximum BTEX threshold is relatively high compared to conventional systems, and may be up to 100, or even up to 500 or up to about 600 ppmv BTEX in the feed gas stream 10. The maximum threshold in addition may depend on CO2 concentration.

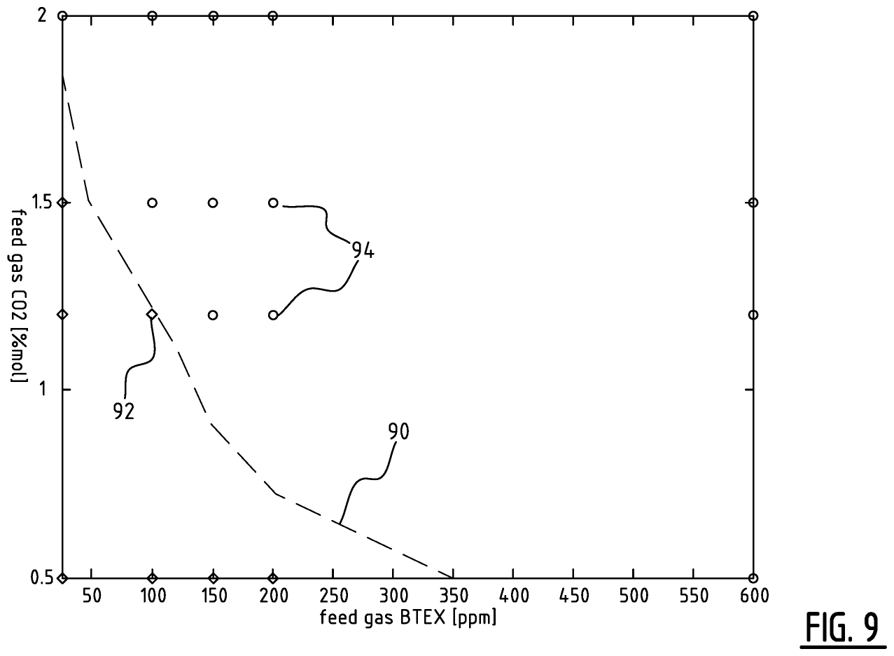

FIGS. 9 to 11 show exemplary diagrams indicating adjustments to the AGRU unit 18. Herein, the horizontal axis indicates BTEX content in the feed gas [expressed in ppm]. The vertical axis indicates CO2 content in the feed gas [expressed in % mol]. Threshold target lines 90, 96, 98 indicate the range wherein the method and system of the disclosure can meet the target threshold for maximum BTEX in the AGRU outlet stream 26. Said threshold may be in the range of 1 to 50 ppm, for instance about 3 to 10 ppm. Dots 92 indicate samples of feed gas composition for which the target threshold for BTEX in the AGRU outlet stream can be met according to specifications. Dots 94 indicate samples of feed gas composition for which the target threshold for BTEX in the AGRU outlet stream is expected to exceed the threshold.

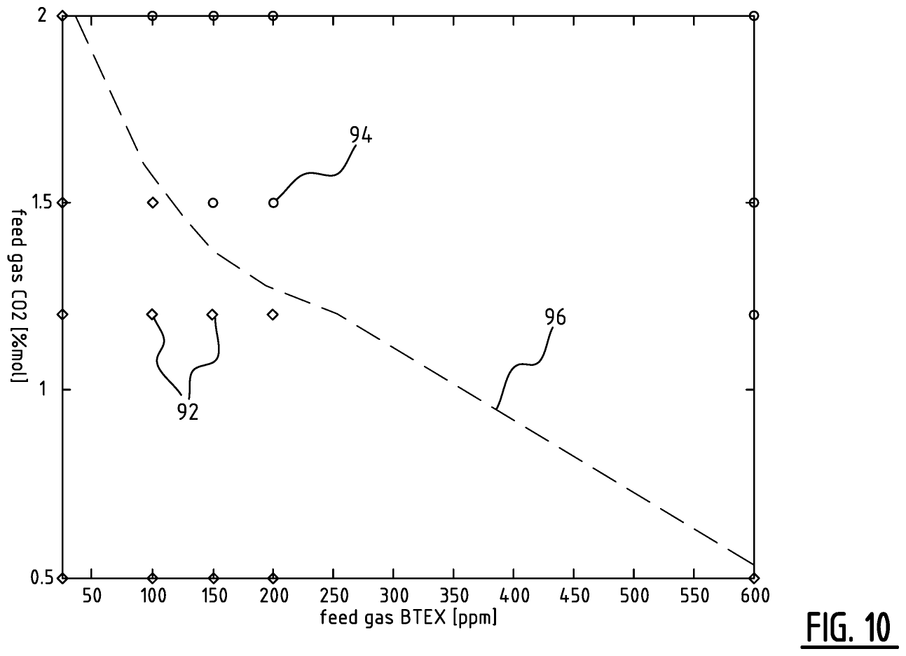

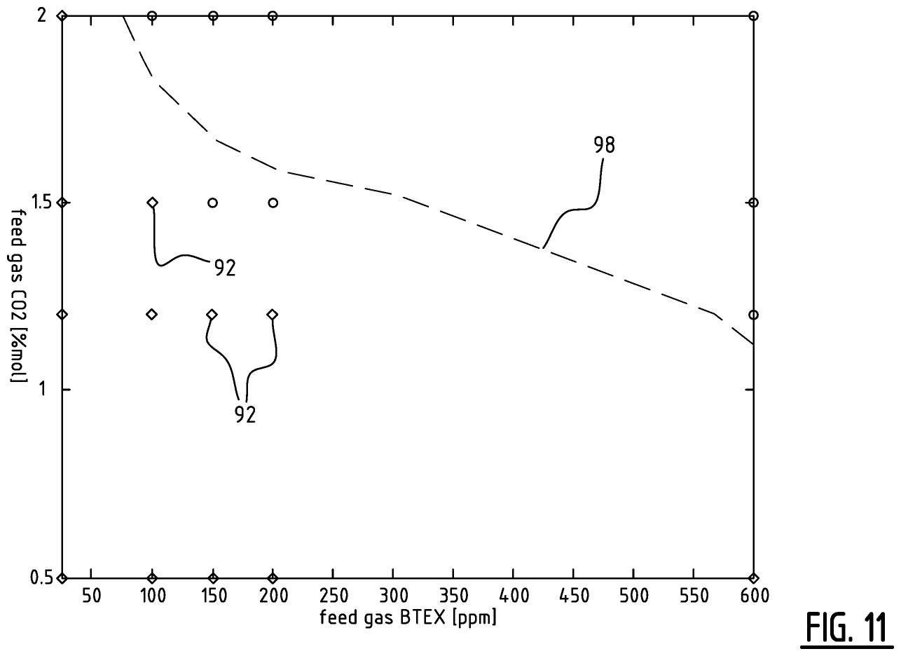

The method of the disclosure allows to adjust the AGRU depending on the feed gas. For instance, assuming the FIG. 10 depicts the system as designed for a certain feed gas composition, including a target line 96 allowing a certain expected variation in feed gas, as covered by dots 92. FIG. 9 shows a scenario wherein for instance the feed gas flow rate increases or the solvent flow rate to the AGRU decreases. Herein, the target threshold line 90 drops with respect to line 96. This allows to lower the solvent flow rate and to save the associated costs for solvent circulation if the feed gas composition and flow rate allow. On the other hand, FIG. 9 also indicates a challenge in case the supply of feed gas increases over time due to changes in market conditions. FIG. 11 indicates a target line 98 covering a wide range of feed gas compositions, wherein the system can process a wide range of feed gas compositions while remaining within preset specifications. Herein, line 98 has an increased window of operating within specifications increased with respect to line 96 in FIG. 10.

The controller 28 can increase the window of operation, i.e. can control the position of the threshold target line at positions shown in FIGS. 9 to 11, for instance increasing the range of feed gas compositions by one or more of increasing the solvent flow rate, lowering the temperature of the optional intercooler 54 in the AGRU, lowering the temperature of the solvent entering the absorber, lowering the feed gas temperature, decreasing the feed gas flow rate and/or changing the solvent composition appropriately. The controller 28 can also decrease the window of operation by the opposite measures, for instance by one or more of decreasing the solvent flow rate, increasing the temperature of the optional intercooler 54 in the AGRU, increasing the temperature of the solvent entering the absorber, increasing the feed gas flow rate of the feed gas 1 and/or changing the solvent composition appropriately.

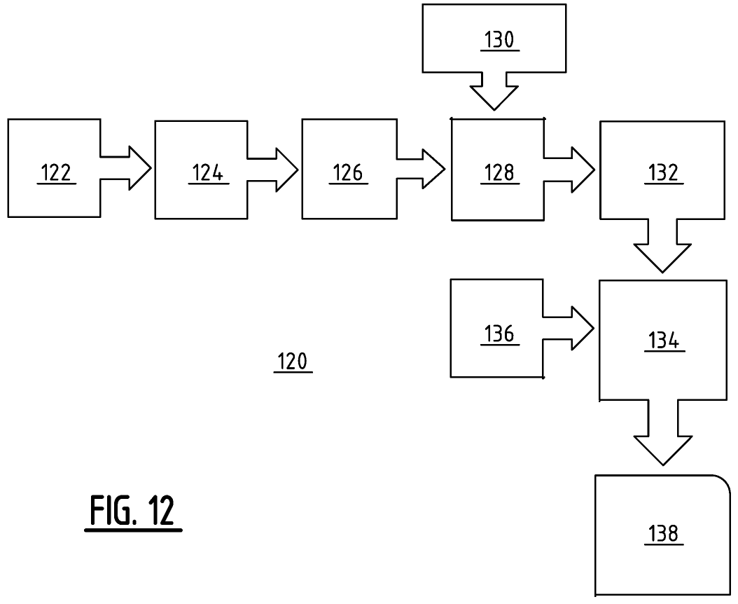

FIG. 12 shows a diagram of an exemplary version of a model 120 suitable to predict removal of aromatic compounds in the AGRU unit.

A first step 122 concerns dedicated thermodynamic lab measurements of hydrocarbon solubility in solvent. The thermodynamic measurements are conducted over a predetermined temperature range. The temperature range is, for instance, about 10 to 100° C. The thermodynamic measurements may be conducted over a predetermined pressure range, for instance, 0.1 to 200 bar. In a practical embodiment, the pressure range is from 0.1 to 60 bar. The measurements may be conducted for a range of solvents, such as ADIP-X and Sulfinol. Typically, the measurements may also be conducted for separate components of Sulfinol, such as an aqueous amine and sulfolane. The data generated in these experiments, or similar thermodynamic measurements, are at present not available in open source data.

A second step 124 concerns the modelling of hydrocarbon solubility in the respective one or more solvents and/or solvent components based on the experimental data resulting from the thermodynamic measurements. The model to describe the thermodynamics is a proprietary model, developed in-house by the applicant.

In a third step 126, the hydrocarbon solubility model of step 124 is included in an overarching rate-based mass transfer model. Said mass transfer model simultaneously solves a heat balance to obtain a converged solution. Examples of mass transfer models are provided in, for instance, Westerterp, K. R., Swaaij, W. P. M. van, Beenackers, A. A. C. M., Chemical Reactor Design and Operation, John Wiley & Sons, 2nd edition, 1982.

In a fourth step 128, the complete model framework is validated against operational data. This operational data is acquired from applicants own full industrial scale operations and is unavailable as open source data.

The validating step 128 is linked with step 130. In step 130, dedicated measurements of AGRU operation at a broad range of operational sites are obtained and provided as data for validation in step 128. As one of the world's largest operators of gas processing sites, Applicant has access to a unique database of operational data to validate the unit process models referenced in the present disclosure. This operational data includes a large set of dedicated field measurements meant for validation of the development of the present disclosure. In addition, the many sites provide deep operational understanding of the process.

In subsequent step 132, a model is derived to describe the impact of process parameters on the removal of soluble components in the Acid Gas Removal Unit 18. Due to the relatively abundant available operational data and the extensive experimental data, the impact of process parameters can be modeled relatively accurately. Relatively accurately herein means, for instance, that the removal of aromatic compounds (such as BTEX) or other contaminants in the AGRU outlet stream can be guaranteed rather than estimated down to tight specifications (such as 4 or 3 ppm or lower).

Subsequent step 134 involves application of a design and control philosophy to the AGRU 18. This step enables to guarantee a maximum output level at the AGRU outlet, for all treated gas specifications within a predetermined range of feed gas composition. Said predetermined range of feed gas composition may include BTEX in the range of 0 to 800 ppm; and/or CO2 in the range of 0 to 3 mol %. Said guarantee may include a maximum threshold of any soluble hydrocarbon component at the AGRU outlet below a certain threshold.

Herein, design philosophy refers to a design guideline or manual which was established specifically for the purpose of removal of contaminants. The guideline takes into account all uncertainties (including operational variations) and manages these uncertainties to obtain a guaranteed performance. It also includes a strict operational window for deployment. The control philosophy means a guideline describing how the AGRU should be operated to meet the guaranteed removal. For instance, margins and tolerances are implemented such, that when the guarantee is a removal up to 3 ppm for each component of BTEX, the actual aim is lower (for instance about 30% lower). For instance, to remove total BTEX down to 3 ppmv, the model may aim for removal down to 2 ppmv or lower (to be able to meet the guarantee under all expected operating conditions).

In practice, removal of each component to a set threshold also means that the total amount of BTEX, i.e. all BTEX components taken together, are guaranteed to be removed to below an only slightly higher set threshold. For instance, for a set threshold of removal of each component to 4 to 0.9 ppmv or lower, the total amount of BTEX will be removed down to, for instance, 5 to 1 ppmv or lower in the AGRU outlet stream. The latter is due to (much) better solubility of BTEX components other than, typically, benzene (benzene typically being the least soluble). This means that the set threshold for benzene ensures that all other components are removed to a (much) lower threshold (some components being removed substantially entirely, down to 0.01-0.001 ppm), so the total of BTEX in the AGRU outlet stream will be the level of benzene (which is guaranteed to be below the set threshold) plus relatively small amounts of the other BTEX components. For instance, benzene may be removed to 3 to 1 ppm or lower, whereas toluene has been removed to 1-0.1 ppm or lower, and all other BTEX components have been removed to trace components (<0.1 ppm or even smaller than 0.01 ppm).

In step 136, experience of operational fluctuations in AGRU operations is used to correct and influence the control philosophy. Again, due to the relatively abundant available operational data available to the Applicants and the extensive experimental data, the impact of operational fluctuations and the effect thereof can be considered relatively accurately.

Step 138 concerns implementation of the above in the AGRU 18. This step results in the capability to deliver the facility depicted in FIG. 1 according to design. Operation of the facility will enable to guarantee that all treated gas within the predetermined range of feed gas composition will be below a set threshold already in the outlet stream 26 of the AGRU. This may include other soluble components of the feed stream, i.e. in addition to aromatic compounds and/or CO2.

The system of the present disclosure allows to handle feed gas having BTEX concentrations up to a concentration where a separate liquid hydrocarbon phase will form in the AGRU absorber 18. This separate liquid phase may form at any location in the absorber where the maximum solubility is reached. This could set an upper limit for the BTEX inlet concentration. This makes AGRU designs without intercooler possible. The intercooler is obviated based on the accuracy of the new model and the underlying correlations.

Due to the improved modelling, the method and system of the present disclosure can guarantee to meet BTEX levels on specification, i.e. below a predetermined maximum threshold, in the AGRU outlet stream 26. Conventional systems only state that if resulting gas from AGRU and subsequent cold flash comprises less than a threshold, such as 3 ppmv BTEX, downstream BTEX removal equipment is obviated. The system of the disclosure can be purposely designed and enables the combined proposed line-up of the present disclosure. The optional features of the line-up will allow choosing the most robust and cost-effective version for a particular feed gas composition and flow rate.

Conventional systems lack a guarantee for C5+ specifications, or at least require additional equipment. Concentrations of C5+ and BTEX are related, and typically there will be C5+ when BTEX is comprised in natural gas. The inclusion of a hydrocarbon wash 88 makes the process of the disclosure more flexible to deal with varying BTEX levels and will decrease the C5+ content in outlet stream 46.

The system and method of the present disclosure obviate the requirement of the depressurization step (i.e. valve 70 in FIG. 8 is optional only).

The system and method of the present disclosure may even obviate the cold flash unit 40. The cold flash 40 removes heavy hydrocarbons, but is not required to remove aromatic compounds such as BTEX. Typically, the benzene specifications will be met in the AGRU outlet stream 26. The model will allow to design for this specification. The controller allows to adjust the AGRU to handle variations in feed gas composition.

The wash step makes the line-up more robust and provides an extra handle to control BTEX. The wash step is in some cases more economical to adjust then to increase the size of the AGRU absorber 18 or to increase the solvent flow rate to the AGRU. Also, the system of the disclosure may obviate the intercooler in the AGRU absorber 18.

As shown in FIGS. 4 and 5, the cryo-flash 40 may be followed by a compressor 60 to compensate for pressure loss and increase subsequent LNG production. The compressor 60 may reduce the penalty for a deep flash. The compressor in combination with the flash unit 40 may render a flash to lower pressure (for instance a pressure drop of about 10 bar or more) economically viable.

| | | | Pressure | |
|---|---|---|---|---|
| Potential | Intercooler 54 | Wash 88 in | reduction 70 | |
| line-up* | in AGRU 17 | cold flash 40 | in cold flash 40 | Comment |
| 1 | no | No | No | Due to modelling and |
| | | | | controller able to meet |
| | | | | BTEX specification in |
| | | | | AGRU outlet stream |
| 2 | no | included | No | More robust than 1. Able to |
| | | | | handle wider range of |
| | | | | impurities in feed. |
| 3** | included | included | No | In case of high CO2 |
| | | | | (>~1.0 mol %) in feed |
| 3** | no | included | included | In case of high BTEX (e.g. |
| | | | | >~300 ppmv) and/or C5 + |
| | | | | (>~1000 ppmv) in feed |
| 4 | included | included | included | All advantages of 1-3. |

*) lowest potential number would be first choice if BTEX and C5+ specifications can be reached. The line-ups will become more expensive as potential number goes up.
**) select depending on feed gas composition.

The system and method of the present disclosure allow more accurate removal of unwanted components in a feed gas stream. Unwanted components herein may include BTEX. For liquefying gas, removal of heavier hydrocarbons, in particular C5+, is an additional aim. The system and method of the present disclosure can guarantee accurate removal of BTEX on specification in the AGRU outlet stream 26. Optionally, the system and method of the present disclosure can guarantee accurate removal of C5+ components on specification in the flash unit outlet stream 46. Specification for BTEX removal herein may be a maximum of any BTEX component, for instance a maximum BTEX concentration set within a range of 1 to 40 ppm. Specification for C5+ removal herein may be a maximum of any C5+ component, for instance set at about 0.1 vol % or lower. As the flash unit 40 may also remove BTEX, the flash gas will meet specifications both for the maximum BTEX concentration and for C5+ content.

The embodiments presented herein obviate requirements for additional equipment after the AGRU. Thus, the system of the disclosure allows much higher removal of BTEX components in the AGRU (for instance up to 98% or even 99.5%) and can guarantee the removal of (total) BTEX already in the AGRU outlet stream. The latter obviates additional downstream equipment to remove contaminants.

The present disclosure enables to guarantee removal of BTEX at the outlet of the AGRU. The removal may include guaranteed removal of C5+, mercaptans, CO2 and/or H2S at the AGRU outlet in conjunction. This means the AGRU is an integral part for achieving the BTEX and C5+ specifications. As a result, downstream units for this purpose can be reduced in size or may be obviated entirely. A typical specification for C5+ is below 500 ppm. Individual components (such as C8) can typically be guaranteed down to 20 ppm or less. Individual components can be guaranteed to 3 ppm or as low as 1 ppm (depending on the feed concentration). Mercaptans are typically guaranteed down to 5 ppm or less, for instance to as low as 3 ppm. The device and method of the present disclosure can allow guaranteed removal of all these components at once down to the respective specifications, in the AGRU outlet stream. This significantly improves reliability of the overall process and facility, as if one of these components exceeds the set specification, production may need to be halted at considerable cost and time.

Scenarios where the improved robustness of the system and method of the present disclosure can provide significant benefits include, for instance:
Fluctuations of the concentration of C5+ and/or BTEX in the feed gas;
Tie in of additional wells (resulting in a change of feed gas composition and flow rate);
High levels of entrainment in the flash vessel; the wash can add robustness;
Operational upsets in the AGRU affecting performance for BTEX removal;
Additional handle to control BTEX concentration out of the line-up.

According to an embodiment, the flash gas 46 may be provided to a liquefaction unit, obviating any further BTEX removal equipment and/or hydrocarbon extraction and separation unit (NGL extraction). The system obviates the requirement to pass the flash gas stream 46 through a scrubbing column, a de-ethanizer or de-methanizer, an adsorber and an extraction unit.

Passing the flash gas to the liquefaction unit, in particular to one or more heat exchangers comprised by the liquefaction unit, may comprise passing the flash gas through a mercury removal unit.

The liquefaction unit (not shown) may comprise a pre-cooling heat exchanger and/or a main cryogenic heat exchanger (MCHE). Both the pre-cooling heat exchanger and/or the main cryogenic heat exchanger may be formed by one or more parallel and/or serial sub-heat exchangers.

The liquefaction unit may use a C3-MR process in which the refrigerant used for the pre-cooling heat exchanger is mainly propane and the refrigerant used for the main cryogenic heat exchanger is a mixed refrigerant. The liquefaction unit may use a DMR process in which the refrigerant used for the pre-cooling heat exchanger is a first mixed refrigerant and the refrigerant used for the main cryogenic heat exchanger is a second mixed refrigerant.

The present disclosure is not limited to the embodiments as described above and the appended claims. Many modifications are conceivable and features of respective embodiments may be combined.

1. A process for producing a purified gas stream from a feed gas stream comprising methane, carbon dioxide, and aromatic compounds selected from the group of benzene, toluene, ethylbenzene, o-xylene, m-xylene and p-xylene (BTEX), the process comprising the steps of:
measuring respective amounts of carbon dioxide and aromatic compounds in the feed gas;
providing the measured amounts of carbon dioxide and aromatic compounds in the feed gas to a controller;
providing the feed gas stream to an acid gas removal unit (AGRU);
providing a stream of absorbing liquid comprising at least sulfolane, water and a secondary or tertiary amine to the AGRU for contacting the feed gas stream with the absorbing liquid in the AGRU;
providing an AGRU waste stream comprising absorbing liquid loaded with carbon dioxide and aromatic compounds;
providing an AGRU outlet stream wherein carbon dioxide and aromatic compounds have been at least partially removed; and
the controller adjusting one or more of composition, temperature, and flow rate of the stream of absorbing liquid to the AGRU to prevent amounts of aromatic compounds in the AGRU outlet stream from exceeding a predetermined maximum threshold.

2. The process of claim 1, comprising the steps of:
measuring respective amounts of carbon dioxide (CO2) and aromatic compounds in the AGRU outlet stream;
providing the measured amounts of carbon dioxide and aromatic compounds in the AGRU outlet stream to the controller; and
the controller adjusting one or more of composition, temperature, and flow rate of the stream of absorbing liquid to the AGRU to prevent said measured amounts of aromatic compounds in the AGRU outlet stream from exceeding the predetermined maximum threshold.

3. The process of claim 1, comprising the step of guaranteeing removal of aromatic compounds down to said predetermined maximum threshold in the AGRU outlet stream.

4. The process of claim 1, comprising the step of guaranteeing an AGRU output stream comprising a total amount of BTEX of 3 or 4 ppmv or less.

5. The process of claim 1, said maximum threshold for aromatic compounds being 3 ppmv benzene and less than 3 ppmv toluene, ethylbenzene and xylene.

6. The process of claim 1, comprising the step of:
creating a model of the AGRU to predict removal of the aromatic compounds from the AGRU outlet stream based on the solubility of the aromatic compounds in the absorbing liquid.

7. The process of claim 6, the model including dependency of the BTEX removal on one of more of absorbing liquid flow rate, absorbing liquid temperature, CO2 content of the feed gas stream, BTEX content in the feed gas, feed gas flow rate, feed gas temperature, AGRU absorber size, and absorbing liquid composition.

8. The process of claim 6, the model including the steps of:
performing thermodynamic measurements of hydrocarbon solubility in the absorbing liquid to provide experimental data;
modelling hydrocarbon solubility in the absorbing liquid based on the experimental data to provide a hydrocarbon solubility model;
including the hydrocarbon solubility model in an overarching rate-based mass transfer model;
obtaining measurements of AGRU operation at one or more operational sites top provide operational measurement data;
validating the mass transfer model against the operational measurement data;
deriving a model describing the impact of process parameters on the removal of soluble components in the AGRU;
applying a design and control philosophy to the AGRU;
correcting the design and control philosophy using operational data; and
implementing the design and control philosophy in the AGRU.

9. The process of claim 1, comprising the step of providing the AGRU outlet stream to a molsieve.

10. The process of claim 9, comprising the steps of:
providing a molsieve outlet stream from the molsieve to a cooler for providing a cooled molsieve outlet stream; and
providing the cooled molsieve outlet stream to a flash unit.

11. The process of claim 10, comprising the step of depressurizing the cooled molsieve outlet stream to provide a depressurized molsieve outlet stream to the flash unit.

12. The process of claim 10, comprising the step of contacting the cooled molsieve outlet stream or the depressurized molsieve outlet stream with a wash liquid in the flash unit to provide a flash gas stream.

13. The process of claim 12, comprising the step of compressing the flash gas stream to provide a compressed flash gas stream.

14. A system for producing a purified gas stream from a feed gas stream comprising methane, carbon dioxide, hydrogen sulfide, and aromatic compounds selected from the group of benzene, toluene, ethylbenzene, o-xylene, m-xylene and p-xylene (BTEX), the system comprising:
a first measurement device for measuring respective amounts of carbon dioxide and aromatic compounds in the feed gas stream;
a controller coupled to the measurement device and arranged to receive the measured amounts of carbon dioxide and aromatic compounds in the feed gas;
an acid gas removal unit (AGRU) for receiving the feed gas stream and adapted for contacting the feed gas stream with a stream of absorbing liquid comprising at least sulfolane, water and a secondary or tertiary amine, the AGRU being adapted to provide an AGRU waste stream comprising absorbing liquid loaded with carbon dioxide and aromatic compounds, and an AGRU outlet stream wherein carbon dioxide and aromatic compounds have been at least partially been removed;
the controller being adapted to adjust one or more of composition temperature, and flow rate of the stream of absorbing liquid to the AGRU to prevent amounts of aromatic compounds in the AGRU outlet stream from exceeding a predetermined maximum threshold.

15. The system of claim 14, comprising:
a second measurement device measuring respective amounts of carbon dioxide and aromatic compounds in the AGRU outlet stream, adapted for providing the measured amounts of carbon dioxide and aromatic compounds in the AGRU outlet stream to the controller.
